(19) United States
(12) Patent Application Publication
(10) Pub. No.: US 2005/0089979 A1
(43) Pub. Date: Apr. 28, 2005

(54) Process for continuous solvent production

(76) Inventors: Thaddeus C. Ezeji, Urbana, IL (US); Nasibuddin Qureshi, Morton, IL (US); Hans P. Blaschek, Champaign, IL (US)

Correspondence Address: GREENLEE WINNER AND SULLIVAN P C, 4875 PEARL EAST CIRCLE, SUITE 200, BOULDER, CO, 80301, US

(21) Appl. No.: 10/945,551

(22) Filed: Sep. 20, 2004

Related U.S. Application Data
(60) Provisional application No. 60/504,280, filed on Sep. 18, 2003.

(51) Int. Cl. C12P 7/28; C12P 7/16; C12P 7/06; C12N 1/21

(52) U.S. Cl. 435/150; 435/161; 435/160; 435/252.3

(57) ABSTRACT

A continuous process for production of solvents, particularly acetone-butanol-ethanol (ABE) using fermentation of solventogenic microorganisms and gas stripping is provided. The solventogenic microorganisms are inoculated in a nutrient medium containing assimilable carbohydrates (substrate) and optional other additives. Control of the solventogenic microorganism concentration in the fermentor (cell concentration) and the assimilable carbohydrate concentration in the fermentor, along with removal of solvents formed results in a continuous process for production of solvents.

### CROSS-REFERENCE TO RELATED APPLICATIONS

This application claims the benefit of U.S. Provisional Application No. 60/504,280, filed Sep. 18, 2003, which application is hereby incorporated by reference to the extent not inconsistent with the disclosure herewith.

### BACKGROUND OF THE INVENTION

The acetone/butanol/ethanol (ABE) fermentation process has received considerable attention in recent years as a method to produce commodity chemicals, such as butanol and acetone, from biomass. The ABE fermentation is the most widely studied among the anaerobic fermentation processes and is a model for complex primary metabolism fermentations.

Butanol is an important industrial chemical. Compared to the currently popular fuel additive ethanol, butanol is more miscible with gasoline and diesel fuel, has a lower vapor pressure, and is less miscible with water, qualities that make butanol a superior fuel extender than ethanol. Butanol is currently used as a feedstock chemical in the plastics industry and as a food grade extractant in the food and flavor industry. Because of the potential for carcinogen carry-over, the use of petroleum-derived butanol is not desirable for food applications.

The fermentation of carbohydrates to acetone, butanol and ethanol by solventogenic microorganisms including clostridia is known. U.S. Pat. No. 5,192,673 describes a fermentation process for producing butanol using a mutant strain of Clostridium acetobutylicum designated Clostridium acetobutylicum ATCC 55025. U.S. Pat. No. 6,358,717, issued Mar. 19, 2002, describes production of solvents using a mutant strain of Clostridium beijerinckii designated Clostridium beijerinckii BA101.

One problem associated with the ABE fermentation by C. acetobutylicum and C. beijerinckii is butanol toxicity to the culture. This toxicity requires continuous removal of the toxic products during the process for maximum production of solvents. Various butanol removal systems, such as pervaporation (Groot et al. 1984), perstraction (Qureshi et al. 1992), reverse osmosis (Garcia et al. 1986), adsorption (Nielson et al. 1988), liquid-liquid extractions (Evans & Wang, 1988), and gas stripping (Groot et al. 1989; Maddox et al. 1995) have been studied by many investigators, but have only been partially successful. There remains a need in the art for an improved process to produce solvents from solventogenic microorganisms.

### BRIEF SUMMARY OF THE INVENTION

A continuous process for production of solvents, particularly acetone/butanol/ethanol (ABE) using fermentation by solventogenic microorganisms and gas stripping is provided. The solventogenic microorganisms are inoculated in a nutrient medium containing assimilable carbohydrates (substrate) and optional other additives. Examples of assimilable carbohydrates are sugars such as glucose, pentose sugars, starch, liquefied starch, enzyme treated liquefied starch, maltodextrin and corn steep liquor. One presently preferred substrate is glucose. Control of the solventogenic microorganism concentration in the fermentor (cell concentration) and the assimilable carbohydrate concentration in the fermentor, along with removal of solvents formed results in a continuous process for production of solvents.

In one embodiment, provided is a method for continuous production of solvents comprising: establishing and maintaining a culture in a fermentor, said culture comprising solventogenic microorganisms and a nutrient medium comprising assimilable carbohydrates and optional other additives; maintaining the assimilable carbohydrate concentration in the fermentor at a level sufficient to maintain a continuous process (in one embodiment between about 20 and 75 g/L); maintaining the concentration of solventogenic microorganisms in the fermentor at a level sufficient to maintain a continuous process; removing solvents from the fermentor by passing a flow of stripping gas through the culture, forming enriched stripping gas; adding water or other suitable liquid to the fermentor to maintain an approximately constant volume in the fermentor; and removing the solvents from the enriched stripping gas. The liquid that is added to the fermentor to maintain an approximately constant volume in the fermentor is any suitable liquid, for example, anaerobically maintained oxygen free water. In one embodiment, the carbohydrate concentration is maintained by adding an assimilable carbohydrate solution having an assimilable carbohydrate concentration of greater than about 250 g/L when the carbohydrate concentration in the fermentor is less than about 20 g/L. Preferably, the assimilable carbohydrate solution added to the fermentor has a carbohydrate concentration between 250 g/L and 500 g/L and all individual values and ranges therein. In one embodiment, the concentration of solventogenic microorganisms in the fermentor is maintained below about 15 g/L (preferably below 10 g/L and more preferably below 7 g/L). In one embodiment, the concentration of solventogenic microorganisms in the fermentor is maintained by removing a portion of the culture when the concentration of solventogenic microorganisms in the fermentor is greater than about 15 g/L by bleeding of the reactor. As known in the art, the concentration of solventogenic microorganisms and carbohydrate concentration required to maintain a continuous process will change as the process is scaled-up. The concentration of solventogenic microorganisms and carbohydrate concentration required to maintain a continuous process are determined using the description herein, along with the knowledge of one of ordinary skill in the art without undue experimentation.

In general the terms and phrases used herein have their art-recognized meaning, which can be found by reference to standard texts, journal references and contexts known to those skilled in the art. The following definitions are provided to clarify their specific use in the context of the invention.

Solventogenic microorganisms are microorganisms that produce solvents. Solventogenic microorganisms include species of Clostridium, including Clostridium beijerinckii and Clostridium acetobutylicum, as well as other microorganisms known in the art. Clostridium beijerinckii BA101 is particularly useful in the present invention because it is a high solvent, low-acid producing strain. Solvents produced by solventogenic microorganisms are known in the art and include a mixture of acetone, butanol and ethanol. In one embodiment, butanol is the major component of the solvent mixture. The production of any combination or ratio of acetone, butanol and/or ethanol using solventogenic microorganisms and gas stripping is included in the invention.

Useful nutrient media include those known to the art, such as P2 and tryptone glucose yeast extract (TGY). Other nutrient media may be used. The nutrient media may optionally contain additives such as salts. Other optional additives are buffers such as PT buffer. P2, TGY and PT may be used and are described in U.S. Pat. No. 6,358,717. Other fermentation conditions such as temperature are easily chosen by one of ordinary skill in the art without undue experimentation. One presently preferred nutrient media is P2 medium.

As used herein, “continuous” means that a measurable amount of solvent is produced while the process is in the “continuous” stage. As known by one of ordinary skill in the art, the culture experiences a different level of solvent production with time, depending on the conditions of the fermentation.

### BRIEF DESCRIPTION OF THE DRAWINGS

FIG. 1 is a diagram of one apparatus useful for continuous ABE production and in-situ recovery by gas stripping. In FIG. 1, Pump A is a feed pump; Pump B is a gas recycle pump; and Pump C is a condensed solvent removal pump.

FIG. 2 shows the concentration of ABE in the reactor described in FIG. 1 during continuous ABE fermentation and recovery by gas stripping in one example fermentation.

FIG. 3A shows a sparger design where holes of increasing diameter are used in a star-shaped form.

FIG. 3B shows a sparger design with a circular-shaped form.

### DETAILED DESCRIPTION OF THE INVENTION

The invention may be further understood by the following non-limiting examples.

The present invention controls the concentration of solventogenic microorganisms (cell concentration) and carbohydrate concentration while removing solvents with gas stripping to achieve a continuous solvent production process using solventogenic microoganisms. The process described herein results in high glucose utilization and a high butanol selectivity as compared to current processes. In particular embodiments, the present invention provides butanol selectivities greater than about 4, greater than about 6 and from 6-11. The process described herein can be an integrated process, where the gas stripping is performed in the same vessel as the solvent production, or the gas stripping can be carried out in a separate vessel connected to the solvent production vessel by the appropriate conduits and connections, as known in the art.

Gas stripping allows for selective removal of volatile substances from a fermentation process without the use of membranes. Gas stripping has been described previously, for example U.S. Pat. No. 4,703,007, issued Oct. 27, 1987; U.S. Pat. No. 4,327,184, issued Apr. 27, 1982; and Qureshi, N., Renewable Energy 22 (2001) 557-564. Generally, gas stripping involves passing a flow of stripping gas through a liquid to form a stripping gas enriched in one or more of the volatile components from the liquid. The volatile components are then removed using means known in the art, for example condensation. The stripping gas used may be any gas that allows recovery of one or more of the volatile components from the fermentation. It is preferred that the stripping gas does not interact with the fermentation process, but as long as the stripping gas does not cause the fermentation to stop, any stripping gas may be used. Some examples of stripping gases include one or more of carbon dioxide, helium, hydrogen and nitrogen. The stripping gases may be a mixture of gases in any desired ratio. One presently preferred stripping gas is a mixture of carbon dioxide and hydrogen. In the examples described herein, the stripping gases were used in the same proportion as they were produced in the fermentor, although other proportions may be used. As known in the art, the flow rate of stripping gas through the liquid in the fermentor is controlled to give the desired level of solvent removal. The particular flow rate of stripping gas through each system is dependent on the configuration of the system, cell concentration and solvent concentration in the reactor, and is determined as known in the art without undue experimentation.

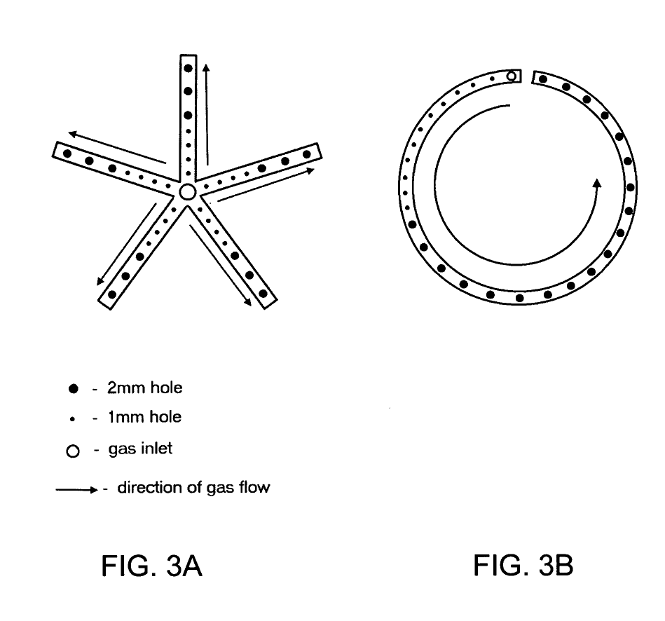

The stripping gas may be passed through the liquid using any desired means, including the use of a sparger such as those illustrated in FIGS. 3A and 3B, and described further below. A source of stripping gas is attached to the sparger by means known in the art.

The gas stripping process has a number of advantages over other extraction processes, for example: (1) it is simple and inexpensive to operate; (2) it does not negatively affect the culture or remove acids from the fermentor; (3) it does not suffer from fouling or clogging by biomass allowing the use of semi-defined substrates, for example; and (4) the stripping gases do not result in extinction of the culture. The gas stripping of the present invention is preferably performed in the fermentor (without a separate external stripping apparatus). However, as known in the art, an external stripping apparatus or other gas stripping configuration may be used.

The following non-limiting description is intended to further illustrate particular embodiments of the invention. One of ordinary skill in the art will recognize that modifications such as a different solventogenic microorganism strain, different nutrient media and additives, different fermentation temperatures, different solventogenic microorganism concentration, different assimilable carbohydrate concentration, different gas stripping apparatus, and different flow rates may be made without undue experimentation. These modifications are intended to be included in the invention.

Organism, culture Maintenance and Fermentation Conditions

C. beijerinckii BA 01 was used for these studies. Spores (200 μl) were heat shocked for 10 min. at 80° C. followed by cooling in an anaerobic chamber for 5 min. The culture was inoculated into 20 ml Tryptone-glucose-yeast extract (TGY) medium (in 50 ml screw capped pyrex bottle) and was incubated anaerobically for 15-16 h at 36±1° C.

The composition of P2 media is as follows: Glucose (60-100 g/L), Yeast extract (1-1.5 g/L), on cooling to 35° C. under oxygen-free nitrogen atmosphere, filter-sterilized P2 stock solutions [(buffer: KH₂PO₄, 50 gL⁻¹; K₂HPO₄, 50 gL⁻¹; Ammonium acetate, 220 gL⁻¹), (vitamin: Para-amino-benzoic acid, 0.1 gL⁻¹; Thiamin, 0.1 gL⁻¹; Biotin, 0.001 gL⁻¹), (mineral: MgSO₄.7H₂O, 20 gL⁻¹; MnSO₄.H₂O, 1 gL⁻¹; FeSO₄.7H₂₀, 1 gL⁻¹; NaCl, 1 gL⁻¹)] were added.

Continuous ABE Fermentation and Gas Stripping

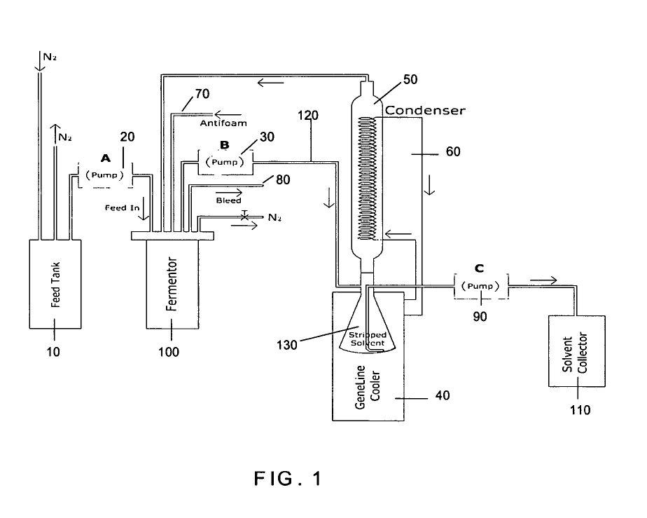

A schematic diagram of one example of the process described herein is shown in FIG. 1. Feed from feed tank (10) is pumped by feed pump (20) into fermentor (bioreactor) (100). In this example, a two-liter bioreactor (New Brunswick Scientific Co., New Brunswick, N.J.) containing P2 medium and glucose at a concentration of 100.3 g/L was inoculated with a 5% (v/v) highly motile cells of C. beijerinckii BA101. The fermentation was allowed to proceed in the batch mode for 24 h. When the ABE concentration approached 4.3 g/L, gas stripping (CO₂ and H₂) was applied, using gases from gas recycle pump (30). Enriched stripping gas is passed through line 120 into condenser (50). The gases were recycled (6000 ml/min) through the system using a twin-head peristaltic pump (30). The cooling machine (40) (for example, GeneLine) was obtained from Beckman Instruments Inc. (Palo Alto, Calif., USA). The ABE vapors were cooled in a condenser (50) (62×600 mm, cooling coil surface area 1292 cm²) to −2° C. using ethylene glycol (60) (50% v/v) circulated at a flow rate of 600 ml/min through the condenser. Oxygen-free distilled water was added into the reactor at intervals to compensate for water loss due to gas-stripping and to maintain a constant liquid level inside the reactor (not shown). Continuous operation was initiated after 48 h at average flow rates of 12 ml/h (feed in) and 6 ml/h (effluent). Addition of antifoam 204 (Sigma Chemicals, St. Louis, Mo., USA) was performed manually through inlet (70) and the temperature was maintained at 35° C. during the entire process. The feed medium was kept anaerobic using oxygen-free nitrogen. Microorganisms were removed through outlet (80) when the concentration exceeded about 10 g/L (range 7-15 g/L). Stripped solvents (130) were removed through pump (90) into collector (110).

Analytical Procedures

Cell concentration was estimated by measuring the optical density. Samples were taken to measure the cell concentration. The sample was centrifuged to remove cell mass followed by suspending the cells in an equal volume of 9 g/L NaCl solution. This procedure was followed in order to remove feed medium components that may interfere with optical density measurement. The suspension was diluted with 9 g/L NaCl solution 10 times followed by measuring optical density using a spectrophotometer. The measured optical density was used to find the dry weight cell concentration using a predetermined correlation between optical density and cell concentration. This cell concentration was multiplied by the dilution factor (10) to obtain the cell concentration in the fermentation broth.

The total amount of ABE produced and acids (acetic and butyric) were measured using a 6890 Hewlett-Packard gas chromatograph (Hewlett-Packard, Avondale, Pa.) equipped with a flame Ionization detector (FID) and 6 ft×2 mm glass column (10% CW-20M, 0.01% H₃PO₄, support 80/100 Chromosorb WAW). The measurement procedure was as follows:

i. Preparation of Acetone-Butanol-Ethanol standard: A) Standard solutions of acetone, butanol and ethanol were prepared with distilled water (acetone 2 g/L, butanol 5 g/L, and ethanol 2 g/L). B) A standard solution (50 g/L) of internal standard (n-propanol) was prepared with distilled water. 1 ml of A and 0.1 ml of B were mixed. 1 μL of the mixture was injected into GC and the peak areas of acetone, butanol, ethanol and n-propanol were shown in the chromatogram. The order of the peaks is acetone →ethanol →n-propanol→butanol. From the peak areas, Response Factors (RF) for each peak was calculated as follows:
For example,

$$\begin{matrix}acetone ( RF ) = \frac{\text{internal standard peak area}}{\text{acetone peak area}} \div \\ \frac{\text{wt of internal standard (5 g)}}{\text{acetone wt (2 g)}}\end{matrix}$$

ii. Preparation of samples for GC analysis: Aliquots of samples were taken from fermentor and centrifuged at 14,000 rpm for 3 min at 4° C. 25 μL of the internal standard was added to 250 μL of the supernatant and mixed. 1 μL of the mixture was injected into GC and the chromatogram displayed the individual ABE peak areas. The concentration of the acetone, butanol or ethanol is calculated as follows:
For example,

$$\text{Conc. in g/L (acetone)} = \frac{\begin{matrix}\text{Wt of internal standard (5 g)} \times \\ RF \times \text{Peak area of acetone}\end{matrix}}{\text{Peak area of internal standard}}$$

(i and ii from personal communication of I.S. Maddox)

Productivity was calculated as total ABE concentration (gL⁻¹) divided by fermentation time (h). Yield was defined as total grams of ABE produced per total grams of glucose utilized. The rate of glucose utilization was defined as the total grams of glucose utilized divided by the fermentation time. Glucose concentration was determined using a hexokinase and glucose-6-phosphate dehydrogenase (Sigma Chemicals, St. Louis, Mo., USA) coupled enzymatic assay. Selectivity (α) is defined as:
α=[y/(1−y)]/[x/(1−x)]
where x is the weight fraction of solvent of interest (for example butanol) in the fermentation broth and y is the weight fraction of solvent of interest in the condensate.
Batch Fermentation

A batch fermentation experiment was run with P2 medium containing 59.9 g/L glucose and using C. beijerinckii BA 01 for comparison. Over the course of 60 h, the culture produced 5.3 g/L acetone, 11.8 g/L butanol, and 0.5 g/L ethanol, resulting in a total ABE concentration of 17.6 g/L (Table 1). The residual glucose at that time was 14.6 g/L. These results demonstrate that the culture was unable to utilize all the glucose because of the toxic effect of butanol. The solvent productivity and yield were 0.29 g L⁻¹ h⁻¹ and 0.39, respectively.

Continuous Fermentation and Recovery by Gas Stripping

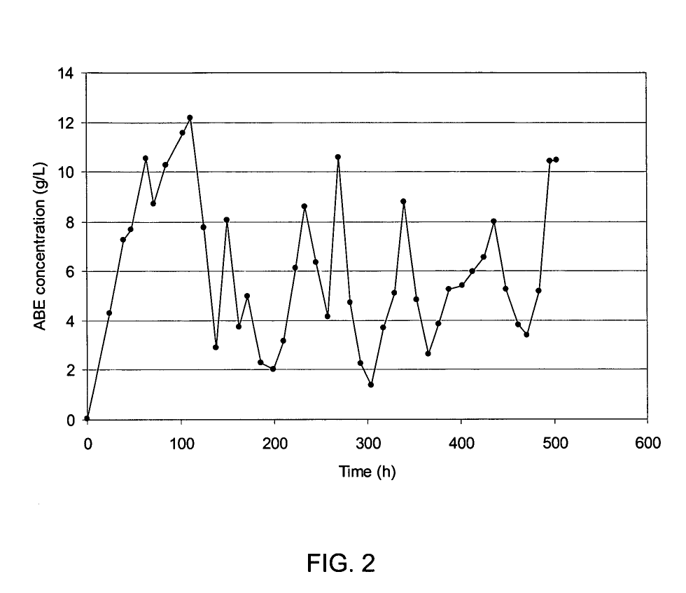

3 g/h glucose was fed into the reactor during fermentation in combination with simultaneous product removal (gas stripping) to increase yield, productivity and reduce process volume. In this study, pH, solvent production, cell and glucose concentrations were monitored. The pH was adjusted to approximately 5.0. The ABE concentration in the reactor is shown in FIG. 2. The total glucose utilized, ABE produced, maximum cell concentration, overall yield and solvent productivity were 1163.4 g/L, 460.4 g/L, 10.4 g/L, 0.39 and 0.91 gL⁻¹ h⁻¹, respectively (Table 1).

TABLE 1
Values of various fermentation parameters in a continuous reactor of an
integrated fermentation product recovery system (gas stripping) using
C. beijerinckii BA101

| | Continuous | Batch |
|---|---|---|
| Parameters | Process | Process |
| Acetone (g/L) | 204.0 | 5.3 |
| Butanol (g/L) | 251.3 | 11.8 |
| Ethanol (g/L) | 5.1 | 0.5 |
| Total ABE (g/L) | 460.4 | 17.6 |
| Fermentation time (h) | 504 | 60 |
| Total glucose utilized (g) | 1163.4 | 45.5 |
| Average glucose utilization rate (g/L/h) | 2.3 | 0.76 |
| Average solvent productivity (g/L/h) | 0.91 | 0.29 |
| Average solvent yield (g/g) | 0.40 | 0.39 |
| Maximum cell concentration (g/L) | 10.4 | 3.51 |
| Maximum ABE concentration in the reactor (g/L) | 12.2 | 17.6 |
| Concentration of glucose in feed (g/L) | 250-500 | 59.9 |

During the continuous ABE fermentation and product recovery by gas stripping, the concentration of carbohydrate in the feed medium was 250-500 g/L. At steady state in one embodiment, the reactor was operated at average flow rates of 12 ml/h (feed in) and 6 ml/h (effluent out). Although the concentration of the carbohydrate in the feed was 250-500 g/L, at this flow rate the carbohydrate become diluted as it enters the fermentor and the bioreactor was able to maintain the concentration of the carbohydrate above 20 g/L necessary to keep the bioreactor functional and below 75 g/L. Above 75 g/L glucose, growth of the culture may be affected due to substrate inhibition. Culture degeneration is usually associated with genetic change and takes place over a period of time, particularly during continuous fermentation. Detailed studies of C. acetobutylicum degeneration during continuous fermentation demonstrated that a population of non-solventogenic Clostridia appears, which coexist for some time with the solventogenic ones, and gradually dominate (Woolley & Morris 1990). In the experiment described here, there was no degeneration and the culture was stable for 21 days after which the process was intentionally terminated.

The integrated continuous system described herein is more energy efficient as concentrated feed substrate (250-500 g/L) is used as opposed to the batch process which uses 60 g/L. Additionally, ABE is removed simultaneously from the reactor thus prolonging life of the reactor. In the continuous system reactor productivity as high as 314% of batch reactor was achieved. This process results in concentrated product stream which requires less energy for further separation and purification. Finally, process streams are reduced, thus making the whole process of ABE production more energy efficient.

ABE Formation in 14-Liter Batch Bioreactor

A 14-liter bioreactor (New Brunswick Scientific Co., New Brunswick, N.J.) was used throughout this study. The bioreactor {(8 L reaction volume), glucose (78.4 g/L) and yeast extract (1 g/L)} were sterilized at 121° C. for 20 min. On cooling to 36° C. under nitrogen atmosphere, filter-sterilized P2 stock solutions were added followed by the inoculation of the bioreactor with 5% (v/v) highly motile cells of C. beijerinckii BA101. Nitrogen was used to sparge the bioreactor until the culture produced enough gases (CO₂ & H₂). Prior to the gas stripping, the condenser and the gas recirculation line were flushed with N₂ to make the system anaerobic. The fermentation was allowed to proceed in the batch mode for 18 h when the ABE concentration was approaching 4.8 g/l after which gas stripping was applied using fermentation gases. Gas stripping was initiated by recycling CO₂ and H₂ through the system (16 Umin), using a twin-head peristatic pump. The ABE vapors were cooled in a condenser to 8° C. Oxygen-free distilled water was added at intervals into the reactor to maintain a constant liquid level (compensate water loss due to gas-stripping) inside the reactor. There was no agitation or pH control and temperature was controlled at 36° C. during the entire process. Antifoam 204 (Sigma chemicals, St. Louis, Mo., USA) was used as an antifoam agent and was added manually. Samples were aseptically withdrawn at intervals for glucose, ABE and optical density analysis. Results are presented in Tables 2 and 3.

TABLE 2
Concentration of ABE and cells (g/L) in the bioreactor (P2 medium)

| | | | | | | | Cell |
|---|---|---|---|---|---|---|---|
| Time | | | | | Acetic | Butyric | Concen- |
| (h) | Acetone | Ethanol | Butanol | ABE | acid | acid | tration |
| 0 | 0.07 | 0.05 | 0.003 | 0.12 | 2.8 | 0.03 | 0.001 |
| 18 | 1.5 | 0.2 | 3.1 | 4.8 | 1.3 | 0.8 | 2.5 |
| 28 | 2.6 | 0.1 | 4.3 | 7.0 | 1.9 | 0.7 | 3.5 |
| 45 | 3.1 | 0.1 | 5.4 | 8.6 | 0.1 | 0.06 | 4.6 |

TABLE 3
Summary of batch ABE production (in P2 medium) in a 14 - liter
bioreactor and recovery by gas stripping

| Parameters | Integrated Batch Fermentation |
|---|---|
| Acetone (g) | 76.4 |
| Butanol (g) | 128.9 |
| Ethanol (g) | 2.8 |
| Total ABE (g) | 208.1 |
| ABE yield (%) | 33.2 |
| Productivity (g/L/h) | 0.58 |
| Initial glucose (g/L) | 78.4 |
| Final glucose (g/L) | 0.0 |
| Fermentation time (h) | 45 |
| Average stripping rate (g/L/h) | 0.64 |
| Total glucose utilized | 627.2 |
| Average stripping rate constant butanol | 0.092 |
| (1/h) | |

Gas stripping was started after 18 h fermentation when the ABE concentration in the bioreactor was 4.8 g/L (Table 2). In this design, at the gas recycle rate of 16 L/min, gas bubbles were created by 100% of the sparger holes due to the reduction of pressure drop between sparger holes. In addition, the gas bubbles created by 2 mm sparger holes enhanced good mixing in the bioreactor during fermentation while the gas bubbles created by 1 mm sparger holes improved the mass transfer (Acetone-butanol removal). The stripping rate constant (Kₛa) for butanol necessary for keeping the butanol concentration below toxic level is 0.059 h⁻¹. In this fermentation a Kₛa of 0.092 h⁻¹ (56% more) was calculated. In addition, the highest concentration of ABE reached in the bioreactor during fermentation and recovery by gas stripping was 8.6 g/L. These show that the rate of ABE removal from the bioreactor was more than the rate of production. The average ABE stripping rate in this experiment was 0.64 g/L.h (Table 3). For these studies the sparger shown in FIG. 3B was used.

Use of liquefied starch and corn steep liquor in ABE fermentation and recovery by gas stripping.

Composition of the Control Medium

Glucose (60 g/L), Yeast extract (1 g/L), on cooling to 35° C. under oxygen-free nitrogen atmosphere, filter-sterilized P2 stock solutions[(buffer: KH₂PO₄, 50 gL⁻¹; K₂HPO₄, 50 gL⁻¹; Ammonium acetate, 220 gL⁻¹), (vitamin: Para-amino-benzoic acid, 0.1 gL⁻¹; thiamin, 0.1 gL⁻¹; Biotin, 0.001 gL⁻¹), (mineral: MgSO₄.7H₂O, 20 gL⁻¹; MnSO₄.H₂₀, 1 gL⁻¹; FeSO₄.7H₂₀, 1 gL⁻¹; NaCl, 1 gL⁻¹)] were added.

The Composition of the Liquefied Starch and Corn Steep Liquor (CSL) Medium:

Liquefied starch (60 g/L), supplied by Archer Daniels Midland (ADM), Decatur, Ill., CSL (16-64 mL/L), on cooling to 35° C. under oxygen-free nitrogen atmosphere, filter-sterilized P2 stock solutions[(buffer: KH₂PO₄, 50 gL⁻¹; K₂HPO₄, 50 gL⁻¹; Ammonium acetate, 220 gL⁻¹), (vitamin: Para-amino-benzoic acid, 0.1 gL⁻¹; thiamin, 0.1 gL⁻¹; Biotin, 0.001 gL⁻¹), (mineral: MgSO₄.7H₂O, 20 gL⁻¹; MnSO₄.H₂₀, 1 gL⁻¹; FeSO₄.7H₂₀, 1 gL⁻¹; NaCl, 1 gL⁻¹)] were added.

The effect of different concentration of corn steep liquor (CSL) on ABE fermentation by C. beijerinckii BA101 was investigated. The importance of this investigation was to substitute substrate glucose and yeast extract with less expensive liquefied starch and CSL. Batch fermentation experiments were run with 16, 32, 48, 56 and 64 ml/L corn steep liquor in 60 g/L liquefied starch medium. After 120 h, the culture produced 9.7, 12.8, 16.9, 18.4 and 18.5 g/L total solvents, respectively, as shown in Table 4. The fermentation time was almost twice that of the control, which shows that the liquefied starch and the corn steep liquor may have an inhibitory effect on C. beijerinckii BA101. However, the culture produced the same amount of ABE as in control when 56-64 g/L CSL was used. Maximum solvent productivity of 0.15 gL⁻¹h⁻¹ was recorded with the 56-64 ml CSL compared to the control with 0.27 gL⁻¹h⁻¹. It is suspected that presence of sodium metabisulfite and other constituents of CSL and liquefied starch are responsible for slow growth of the culture, which might affect amylolytic enzyme production and result in poor starch/oligosaccharides utilization.

Liquefied starch was hydrolyzed with glucoamylase enzyme and the effect of different corn steep liquor (CSL) concentration on ABE fermentation by C. beijerinckii BA 01 was investigated. The total solvent produced by the culture is similar to the amount it produced with liquefied starch. However, the fermentation time was reduced to 78 h and the maximum solvent productivity was increased to 0.23-0.24 gL⁻¹ h⁻¹ (Table 5). The fermentation time for the control was 68 h, which shows that the fermentation was faster in the control than in both the liquefied and hydrolyzed liquefied starch fermentations.

The effect of liquefied starch and CSL on batch ABE fermentation and recovery by gas stripping was also investigated. In order to allow cell growth, stationary fermentation was allowed for 15 h and 24 h for control and liquefied starch, respectively. At that stage ABE recovery by gas stripping was started and the fermentation was allowed to run for 39 h for the control at which time glucose concentration was reduced to 0 g/L. It is important to note that at the end of fermentation and recovery, acids were not detected either in the reactor or in the condensate, suggesting that the system became truly solventogenic. This was not the case when liquefied starch and CSL were used. However, liquefied starch and CSL produced a higher total ABE and a better yield than the control due to the extra carbon source contributed by CSL. Other constituents of the liquefied starch and CSL such as sulfite, lactic and phytic acids, heavy metals and their salts may have inhibited C. beijerinckii BA101 during the early stage of the fermentation, thereby increasing the fermentation time up to 67-81 h and residual acids accumulation in the fermentor. These results are shown in Table 6.

TABLE 4
Effect of liquefied starch and corn steep liquor (CSL) on ABE fermentation by
C. beijerinckii BA 101.

| | Control | | | | | |
|---|---|---|---|---|---|---|
| | [glucose | 16 mL | 32 mL | 48 mL | 56 mL | 64 mL |
| Parameters | 60 gL⁻¹] | CSL | CSL | CSL | CSL | CSL |
| Acetone [gL⁻¹] | 4.4 | 1.91 | 2.1 | 3.9 | 4.3 | 4.1 |
| Butanol [gL⁻¹] | 13.7 | 7.51 | 10.3 | 12.3 | 13.4 | 13.8 |
| Ethanol [gL⁻¹] | 0.5 | 0.28 | 0.4 | 0.7 | 0.7 | 0.6 |
| Total ABE [gL⁻¹] | 18.6 | 9.7 | 12.8 | 16.9 | 18.4 | 18.5 |
| ABE productivity [gL⁻¹h⁻¹] | 0.27 | 0.10 | 0.11 | 0.14 | 0.15 | 0.15 |
| Acetic acid [gL⁻¹] | 1.2 | 1.0 | 1.0 | 0.5 | 0.7 | 1.7 |
| Butyric acid [gL⁻¹] | 0.7 | 0.5 | 0.5 | 0.3 | 0.3 | 0.3 |
| Total acids [gL⁻¹] | 1.9 | 1.5 | 1.5 | 0.8 | 1.0 | 2.0 |
| Fermentation time [h] | 68 | 96 | 120 | 120 | 120 | 120 |

TABLE 5
Effect of different concentrations of corn steep liquor on ABE fermentation using
glucoamylase treated liquefied starch [60 gL⁻¹] as substrate

| | | 16 mL | 32 mL | 48 mL | 56 mL | 64 mL |
|---|---|---|---|---|---|---|
| | Control | corn | corn | corn | corn | corn |
| | [glucose | steep | steep | steep | steep | steep |
| Parameters | 60 gL⁻¹] | liquor | liquor | liquor | liquor | liquor |
| Acetone [gL⁻¹] | 4.4 | 2.9 | 3.6 | 3.9 | 4.0 | 3.8 |
| Butanol [gL⁻¹] | 13.7 | 9.8 | 12.8 | 13.2 | 13.4 | 13.9 |
| Ethanol [gL⁻¹] | 0.5 | 0.5 | 0.8 | 0.9 | 0.8 | 0.9 |
| Total ABE [gL⁻¹] | 18.6 | 13.2 | 17.2 | 18.0 | 18.2 | 18.6 |
| ABE productivity [gL⁻¹h⁻¹] | 0.27 | 0.19 | 0.22 | 0.23 | 0.23 | 0.24 |
| Acetic acid [gL⁻¹] | 1.2 | 2.2 | 3.9 | 3.8 | 1.8 | 3.0 |
| Butyric acid [gL⁻¹] | 0.7 | 0.6 | 1.3 | 0.4 | 0.2 | 0.6 |
| Total acids [gL⁻¹] | 1.9 | 2.8 | 5.2 | 4.2 | 2.0 | 3.6 |
| Fermentation time [h] | 68 | 68 | 78 | 78 | 78 | 78 |

TABLE 6
Effect of liquefied and glucoamylase treated liquefied starch and
CSL on ABE fermentation by C. beijerinckii BA 101 and recovery
by gas stripping.

| | | 60 g/L liq. | 60 g/L | 60 g/L |
|---|---|---|---|---|
| | *Control | starch + | glucoamylase | glucoamylase |
| | [glucose] | 56 mL | liq. starch + | liq. starch + |
| Parameters | 60 gL⁻¹ | CSL | 56 mL CSL | yeast extract |
| Acetone [gL⁻¹] | 6.9 | 7.7 | 8.3 | 9.5 |
| Butanol [gL⁻¹] | 16.4 | 15.1 | 17.6 | 16.4 |
| Ethanol [gL⁻¹] | 0.3 | 1.1 | 0.6 | 0.4 |
| Total ABE | 23.6 | 23.9 | 26.5 | 26.3 |
| [gL⁻¹] | | | | |
| ABE | 0.61 | 0.31 | 0.40 | 0.39 |
| productivity | | | | |
| [gL⁻¹h⁻¹] | | | | |
| Yield [g/g] | 0.40 | 0.40 | 0.44 | 0.44 |
| Acetic acid | 0.0 | 2.9 | 1.2 | 1.0 |
| [gL⁻¹] | | | | |
| Butyric acid | 0.0 | 1.9 | 1.0 | 0.7 |
| [gL⁻¹] | | | | |
| Total acids | 0.0 | 4.8 | 2.2 | 1.7 |
| [gL⁻¹] | | | | |
| Fermentation | 39 | 78 | 67 | 67 |
| time [h] | | | | |

*No CSL and liquefied starch

Liquefied starch and CSL can be used to produce ABE. Fermentation of liquefied starch (60 g/L) supplemented with 56-64 ml CSL solution in a batch process resulted in the production of 18.4 g/L of ABE, similar to the control experiment (18.6 g/L ABE). ABE was produced in a batch fermentor integrated with gas stripping product removal system. In the batch process with recovery, the total ABE produced was above 26 g/L from 60 g/L of liquefied starch. The yield was found to be 0.44 with an average productivity of 0.4 gL⁻¹h⁻¹. Liquefied starch and CSL are useful for bioconversion of starch to ABE. However, the presence of sodium metabisulfite (Na₂S₂O₅) in the liquefied starch and CSL is a problem in the fermentation by C. beijerinckii BA101. Concentrations as low as 0.2 g/L Na₂S₂O₅ in the fermentation broth exerts inhibition on C. beijerinckii BA101 and growth seizes when the concentration exceeds 0.5 g/L. Sodium metabisulfite concentrations of the liquefied starch and CSL were found to be 0.71 and 1.31 g/L, respectively. However, liquefied starch and CSL were diluted seven and fifteen times, respectively by the time they are ready to be inoculated with C. beijerinckii BA101 culture. The results presented in Tables 4-6 demonstrate that substrates and nutrient media other than glucose and yeast extract based medium (semi-synthetic medium) can effectively be used in this integrated butanol fermentation and recovery system, thus making the process of butanol production more economically attractive. It also shows that this process is not limited to the use of pure glucose and expensive nutrients.

Sparger Design

The sparger is used to pass a stripping gas or gasses through the fermentation process. The stripping gas is passed into the sparger, which may be a perforated ceramic or other design. The solvents are then stripped from the stripping gas using methods known in the art.

Some examples of different sparger designs that are useful in the practice of the invention, as well as for other uses known in the art are shown in FIG. 3. In FIG. 3, the arrows show gas flow direction. Provided is an apparatus for formation of gas bubbles comprising one or more gas flow arms having one or more gas flow holes, said one or more gas flow arms attached to a central gas inlet, said central gas inlet connected to a gas source. The gas flow arms may be arranged in the same horizontal plane, such as that shown in FIG. 3A, or may be arranged in different horizontal planes. It is preferred that the gas flow arms arranged equidistant from each other, although that is not required. In one embodiment, the one or more gas flow arms are arranged in a star-shaped form. In another embodiment, there is one gas flow arm which is curved in shape. A particular embodiment shown in FIG. 3B shows one gas flow arm which is circular in shape.

FIG. 3A shows a design where the stripping gas flows into the center of a star-shaped form. The star-shaped form has holes from which the stripping gas flows. In the example shown in FIG. 3A, the star-shaped form has five arms. As known in the art, more or fewer arms may be used. More arms provide more stripping gas contact with the fermentation broth. In the example shown in FIG. 3A, the diameter of holes increases as the gas flows away from the gas inlet. This is to allow gas to escape from the entire length of the gas flow arms, rather than all of the stripping gas to escape near the gas inlet. However, it is not required that the diameter of holes increases as the gas flows away from the gas inlet in any sparger design, as long as a sufficient amount of stripping gas is passed through the fermentation broth to produce the desired amount of solvents.

FIG. 3B shows another embodiment of a sparger design. In FIG. 3B, gas flows around a circular gas flow arm. As known in the art, other geometric forms can be used, such as square or rectangular, however, since the fermentor is usually round, the circular design is particularly useful to pass the stripping gas through the fermentor. The flow rate may be adjusted as known in the art to provide the desired amount of gas flow through the sparger. In one example, the gas flow rate is 20 to 25 L/min.

While decreasing bubble size will increase the rate of mass transfer per unit volume of gas, it does not increase the saturation concentration of butanol in the gas phase. Having smaller holes near the gas inlet will increase the number of gas bubbles in the bioreactor with possible reduction of pressure drop in the sparger holes. Reduction or elimination of pressure drop in the sparger will enhance gas bubbles formation from all the sparger holes at a reduced gas recycle rate.

Scale Up

The process described herein can be easily scaled up to a larger scale by suitable selection of reactor size, condenser size and other apparatus modifications, as known to one of ordinary skill in the art without undue experimentation. The solventogenic microorganism concentration, carbohydrate concentration and other parameters are adjusted to provide a continuous process, using the description herein, as well as the knowledge of one of ordinary skill in the art without undue experimentation.

Automation

The operation of the process described herein can be automated using a microprocessor system. For example, the system can monitor the cell concentration, compare the cell concentration to a predetermined value, and remove a portion of the culture when the cell concentration is greater than a predetermined value. The system can also add water or other additives to maintain a predetermined volume in the fermentor. The system can monitor the carbohydrate concentration, compare the carbohydrate concentration to a predetermined value, and if the carbohydrate concentration is lower than the predetermined value, add carbohydrate solution.

When a group of substituents is disclosed herein, it is understood that all individual members of those groups and all subgroups and classes of compounds that can be formed using the substituents are disclosed separately. When a Markush group or other grouping is used herein, all individual members of the group and all combinations and subcombinations possible of the group are intended to be individually included in the disclosure.

Every combination of components described or exemplified can be used to practice the invention, unless otherwise stated. Specific names of compounds and components such as microorganisms are intended to be exemplary, as it is known that one of ordinary skill in the art can name the same compounds differently. One of ordinary skill in the art will appreciate that methods, device elements, starting materials, synthetic methods, and microorganism growth conditions other than those specifically exemplified can be employed in the practice of the invention without resort to undue experimentation. All art-known functional equivalents, of any such methods, device elements, starting materials, synthetic methods, and microorganism growth conditions are intended to be included in this invention. Whenever a range is given in the specification, for example, a temperature range, a time range, or a composition range, all intermediate ranges and subranges, as well as all individual values included in the ranges given are intended to be included in the disclosure to the same extent as if they were individually listed.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

One skilled in the art would readily appreciate that the present invention is well adapted to carry out the objects and obtain the ends and advantages mentioned, as well as those inherent therein. The solventogenic microorganisms and methods and accessory methods described herein as presently representative of preferred embodiments are exemplary and are not intended as limitations on the scope of the invention. Changes therein and other uses will occur to those skilled in the art, which are encompassed within the spirit of the invention, are defined by the scope of the claims.

Although the description herein contains many specificities, these should not be construed as limiting the scope of the invention, but as merely providing illustrations of some of the embodiments of the invention. Thus, additional embodiments are within the scope of the invention and within the following claims. All references cited herein are hereby incorporated by reference to the extent that there is no inconsistency with the disclosure of this specification. Some references provided herein are incorporated by reference herein to provide details concerning additional nutrient media, solventogenic microorganisms, gas stripping details, additional methods of analysis and additional uses of the invention.

### REFERENCES

Atkinson, B. and Mavintuna, F. 1983. Biochemical engineering and biotechnology Handbook, Nature Press. In: Harvey W. Blanch and Douglas S. Clark. Biochemical Engineering. Marcel Dekker Inc. New York. pp.169.
Busche, R. M, and Allen B. R. 1989. Technoeconomics of butanol extractive fermentation in a multiphase fluidized bed bioreactor. Appl. Biochem. Biotechnol. Vol. 20/21: 357-374.
Evans P J and H W Wang. 1988. Enhancement of butanol fermentation by Clostridium acetobutylicum in the presence of decanol-oleyl alcohol mixed extractants. Appl. Environ. Microbiol. 54:1662-1667.
Garcia A, E L Ianotti, and J L Fischer. 1986. Butanol fermentation liquor production and separation by reverse osmosis. Biotechnol. Bioeng. 28:785-791.
Groot W J, C E van den Oever, and N W F Kossen. 1984. Pervaporation for simultaneous product recovery in the butanol/isopropanol batch fermentation. Biotechnol. Lett. 6:709-714.
Groot W J, R G J M van der Lans, and KChAM Luyben. 1989. Batch and continuous butanol fermentation with free cells: integration with product recovery by gas stripping. Appl. Microbiol. Biotechnol. 32:305-308.
Maddox I S, N Qureshi and K Roberts-Thomson. 1995. Production of acetone butanol ethanol from concentrated substrates using Clostridium acetobutylicum in an integrated fermentation-product removal process. Process Biochemistry 30(3):209-215.
Nielson L, M Larsson, 0 Holst, and B Mattiasson. 1988. Adsorbents for extractive bioconversion applied to the acetone butanol fermentation. Appl. Microbiol. Biotechnol. 28:335-339.
Qureshi N and H P Blaschek. 2001. Recovery of butanol from fermentation broth by gas stripping. Renewable Energy 22:557-564.
Qureshi N, I S Maddox, and A Friedl. 1992. Application of continuous substrate feeding to the ABE fermentation: Relief of product inhibition using extraction, perstraction, stripping, and pervaporation. Biotechnol. Prog. 8:382-390.
Wooly R C and J G Morris. 1990. Stability of solvent production by Clostridium acetobutylicum in continuous culture: strain differences. J. Appl. Bacteriol. 69:718-728. U.S. Pat. Nos. 5,036,005; 5,215,902; 4,510,242; 4,424,275; 5,063,156; 4,568,643; 4,520,104; 4,568,643; 4,703,007; 4,560,658; 4,327,184.

1. A continuous process for production of solvents comprising:
establishing and maintaining a culture in a fermentor, said culture comprising solventogenic microorganisms and a nutrient medium comprising assimilable carbohydrates and optional additives;
maintaining the carbohydrate concentration in the fermentor at a level sufficient to maintain a continuous process;
maintaining the concentration of solventogenic microorganisms at a level sufficient to maintain a continuous process;
removing solvents from the fermentor by passing a flow of stripping gas through the culture, forming enriched stripping gas;
adding water or other suitable liquid to the fermentor to maintain an approximately constant volume in the fermentor; and
removing the solvents from the enriched stripping gas.

2. The process of claim 1, wherein said solventogenic microorganisms are selected from the group consisting of: Clostridium beijerinckii and Clostridium acetobutylicum.

3. The process of claim 2, wherein said solventogenic microorganisms are Clostridium beijerinckii BA101.

4. The process of claim 1, wherein said stripping gas is recirculated to the fermentor after the solvents are removed.

5. The process of claim 1, wherein the solvents are removed from the enriched stripping gas by cooling the enriched stripping gas to condense the solvents and collecting the condensed solvents.

6. The method of claim 1, wherein the solvents comprise one or more of acetone, butanol and ethanol.

7. The process of claim 1, wherein the stripping gas comprises carbon dioxide and hydrogen.

8. The process of claim 1, wherein the assimilable carbohydrate is glucose.

9. The process of claim 1, wherein the nutrient medium comprises corn steep liquor, starch and optional additives.

10. The process of claim 1, wherein the nutrient medium is semi-synthetic.

11. The process of claim 1, wherein the carbohydrate concentration in the fermentor is maintained between about 20 and 75 g/L.

12. The process of claim 11, wherein the carbohydrate concentration in the fermentor is maintained by adding a carbohydrate solution having a carbohydrate concentration of greater than about 250 g/L when the carbohydrate concentration in the fermentor is less than about 20 g/L.

13. The process of claim 1, wherein the concentration of solventogenic microorganisms in the fermentor is maintained below about 15 g/L.

14. The process of claim 13, wherein the concentration of solventogenic microorganisms in the fermentor is maintained by removing a portion of the culture when the concentration of solventogenic microorganisms in the fermentor is greater than about 15 g/L.

15. A continuous process for production of solvents comprising:
establishing and maintaining a culture in a fermentor, said culture comprising solventogenic microorganisms and a nutrient medium comprising assimilable carbohydrates and optional additives;
monitoring the carbohydrate concentration in the fermentor;
maintaining the carbohydrate concentration in the fermentor at a level sufficient to maintain a continuous process;
monitoring the concentration of solventogenic microorganisms in the fermentor;
maintaining the concentration of solventogenic microorganisms in the fermentor at a level sufficient to maintain a continuous process;
removing solvents from the fermentor by passing a flow of stripping gas through the culture, forming enriched stripping gas;
adding water or other suitable liquid to the fermentor to maintain an approximately constant volume in the fermentor; and
removing the solvents from the enriched stripping gas.

16. The process of claim 15, wherein said solventogenic microorganisms are selected from the group consisting of: Clostridium beijerinckii and Clostridium acetobutylicum.

17. The process of claim 16, wherein said solventogenic microorganisms are Clostridium beijerinckii BA101.

18. The process of claim 15 wherein said stripping gas is recirculated to the fermentor after the solvents are removed.

19. The process of claim 15, wherein the solvents are removed from the enriched stripping gas by cooling the enriched stripping gas to condense the solvents and collecting the condensed solvents.

20. The method of claim 15, wherein the solvents comprise one or more of acetone, butanol and ethanol.

21. The process of claim 15, wherein the stripping gas comprises carbon dioxide and hydrogen.

22. The process of claim 15, wherein the assimilable carbohydrate is glucose.

23. The process of claim 15, wherein the nutrient medium comprises corn steep liquor, starch and optional additives.

24. The process of claim 15, wherein the carbohydrate concentration in the fermentor is maintained between about 20 and 75 g/L.

25. The process of claim 24, wherein the carbohydrate concentration in the fermentor is maintained by adding a carbohydrate solution having a carbohydrate concentration of greater than about 250 g/L when the carbohydrate concentration in the fermentor is less than about 20 g/L.

26. The process of claim 15, wherein the concentration of solventogenic microorganisms in the fermentor is maintained below about 15 g/L.

27. The process of claim 26, wherein the concentration of solventogenic microorganisms in the fermentor is maintained by removing a portion of the culture when the concentration of solventogenic microorganisms in the fermentor is greater than about 15 g/L.

28. A continuous process for production of solvents comprising:
establishing and maintaining a culture in a fermentor, said culture comprising solventogenic microorganisms and a nutrient medium comprising assimilable carbohydrates and optional additives;
maintaining the carbohydrate concentration in the fermentor at between about 20 and 75 g/L by adding a carbohydrate solution having a carbohydrate concentration of greater than about 250 g/L when the carbohydrate concentration in the fermentor is less than about 20 g/L;
maintaining the concentration of solventogenic microorganisms at below about 15 g/L by removing a portion of the culture when the concentration of solventogenic microorganisms in the fermentor is greater than about 15 g/L;
removing solvents from the fermentor by passing a flow of stripping gas through the culture, forming enriched stripping gas;
adding water or other suitable liquid to the fermentor to maintain an approximately constant volume in the fermentor; and
removing the solvents from the enriched stripping gas.

29. An apparatus for formation of gas bubbles comprising one or more gas flow arms having one or more gas flow holes, said one or more gas flow arms attached to a central gas inlet, said central gas inlet connected to a gas source.

30. The apparatus of claim 29 wherein the one or more gas flow arms are arranged in the same horizontal plane, said arms arranged equidistant from each other.

31. The apparatus of claim 30, wherein the one or more gas flow arms are arranged in a star-shaped form.

32. The apparatus of claim 29, comprising one gas flow arm which is curved in shape.

33. The apparatus of claim 32, comprising one gas flow arm which is circular in shape.
